(19) United States
(12) Patent Application Publication
(10) Pub. No.: US 2026/0157689 A1
(43) Pub. Date: Jun. 11, 2026

(54) SYSTEMS AND METHODS FOR NON-INVASIVE DETECTION OF SLEEP STAGES

(71) Applicant: THE TRUSTEES OF INDIANA UNIVERSITY, Bloomington, IN (US)

(72) Inventors: Thomas H. Everett, Indianapolis, IN (US); Md Aktaruzzaman, Indianapolis, IN (US)

(21) Appl. No.: 19/181,513

(22) Filed: Apr. 17, 2025

Related U.S. Application Data
(60) Provisional application No. 63/635,789, filed on Apr. 18, 2024.

(51) Int. Cl. A61B 5/00; A61B 5/0205; A61B 5/318; A61B 5/388

(52) U.S. Cl. CPC A61B 5/4812; A61B 5/0205; A61B 5/318; A61B 5/388; A61B 5/7267

(57) ABSTRACT

Systems and methods are provided for using skin nerve activity and heart activity to identify a plurality of sleep stages of a subject. The skin nerve activity and heart activity can be detected using a sensor that can be part of wearable clothing or a clothing accessory, including watches and jewelry. From the monitored skin nerve activity and heart activity, one or more activity features can be extracted that can be used by a machine learnable model to characterize the different stages of sleep. Further, the particular activity features that are to be extracted can be identified by the machine learnable model, and can comprise less than all the possible extractable activity features. Moreover, the extracted activity features can be based on the influence those features, or the associated signatures, have on indicating each of the different stages of sleep.

### CROSS-REFERENCE TO RELATED APPLICATIONS

The present application claims priority to U.S. Provisional Patent Application No. 63/635,789, filed on Apr. 18, 2024, the contents of which are hereby incorporated by reference in their entirety.

### REFERENCE TO GOVERNMENT GRANTS

This invention was made with government support under HL158952 awarded by National Institutes of Health. The Government has certain rights in the invention.

### TECHNICAL FIELD

The present disclosure generally relates to detecting and characterizing different stages of sleep, and more particularly, but not exclusively, to an automated system and process that incorporates multi-modal wearable sensor technologies and artificial intelligence to identify unique nerve activity and heart rate variability signatures in characterizing different stages of sleep.

### BACKGROUND

Sleep is a complex dynamical process and can be important for building and recovery of human body, bone, and memory. During normal sleep conditions, a sleeping person can move into five basic sleep stages: non-rapid eye movement (NREM-1, NREM-2, NREM-3), rapid eye movement (REM), and awake (WAKE). Each of these five sleep stages can play a significant role in physiological recovery. For example, the non-rapid eye movement sleep stage associated with deep sleep (NREM-3) can contribute to insightful thinking, repair injuries, and reinforce immune system. On the other hand, the REM sleep stage is believed to be essential to cognitive functions like memory, learning, and creativity. Further, poor quality of sleep can be associated with impaired functioning, including, for example, an increased chance of a motor vehicle accident and reduced job performance or productivity, as well as be linked to certain adverse health outcomes, including, for example, an increased risk of cardiovascular disease, obesity, and neurodegenerative disorders.

Currently, chronic sleep disorders are estimated to affect approximately millions of Americans. One such chronic sleep disorder is sleep apnea, which is a complete cessation of airflow for at least ten (10) seconds while sleeping. Yet, most of these cases often go undiagnosed and untreated. While multiple factors can contribute to the large number of undiagnosed cases, the cost and inconvenience associated with the execution of a polysomnogram for diagnosing sleep apnea are often identified as reasons for the lack of diagnosis. Moreover, polysomnograms historically have required patients to schedule an overnight stay at a sleep clinic and can be costly. For example, such testing historically is to take place using specialized, and expensive, equipment in the presence of trained personnel or sleep experts at a laboratory. Yet, outcomes of the assessments are often highly variable due to inconsistencies in sleep patterns and subjective interpretation of the results. The results can also be adversely impacted by the subject not exhibiting normal sleep behavior while trying to sleep in a new setting, namely in the laboratory. Additionally, it can take several months before the polysomnogram can be performed, which lengthens the diagnostic process for both sleep apnea and patients that present with similar symptoms but require other treatments. Further, once therapy is prescribed for sleep apnea, verifying its effectiveness requires continuous patient follow-up within the first few weeks of initiating treatment.

### SUMMARY

The present disclosure can comprise one or more of the following features and combinations thereof.

In one embodiment of the present disclosure, a system is provided for identifying a plurality of sleep stages of a subject. The system can include a monitor having at least one sensor configured to obtain a captured information regarding a skin nerve activity and a heart activity of the subject, at least one processor, and a memory coupled with the at least one processor. The memory including instructions that when executed by the at least one processor can cause the at least one processor to: determine a plurality of activity features from the captured information regarding both the skin nerve activity and the heart activity of the subject; generate one or more signals to facilitate the determined plurality of activities being used with a machine learnable model to identify one or more sleep stages of the plurality of sleep stages; and/or generate one or more signals to facilitate a communication of the identified one or more sleep stages to the subject.

In another embodiment, a method is provided for identifying a plurality of sleep stages of a subject. The method can include capturing, using at least one sensor, a captured information regarding a skin nerve activity and a heart activity of the subject, and can also include determining a plurality of activity features from the captured information regarding both the skin nerve activity and the heart activity of the subject. Additionally, the method can include generating one or more signals to facilitate the plurality of activity features being used with a machine learnable model to identify one or more sleep stages of the plurality of sleep stages, and/or generating one or more signals to facilitate a communication of the identified one or more sleep stages to the subject.

These and other features of the present disclosure will become more apparent from the following description of the illustrative embodiments.

### BRIEF DESCRIPTION OF THE FIGURES

The disclosure described herein is illustrated by way of example and not by way of limitation in the accompanying figures. For simplicity and clarity of illustration, elements illustrated in the figures are not necessarily drawn to scale. For example, the dimensions of some elements can be exaggerated relative to other elements for clarity. Further, where considered appropriate, reference labels have been repeated among the figures to indicate corresponding or analogous elements.

FIG. 1 illustrates a simplified block diagram of a system for characterizing different stages of sleep utilizing information captured from the skin of a subject.

FIG. 2 illustrates an exemplary method 200 for training and developing first and second models based on an activity feature set, and a selected subset of the activity features, respectively derived from ECG and SKNA measurements for characterizing different stages of sleep of a subject.

FIG. 3 illustrates an exemplary method for utilizing the first and second models identified in FIG. 2 to generate a determination of stages of sleep of the subject.

FIG. 4 illustrates the sleep hypnogram from a study in mean values of the R-R intervals and skin sympathetic nerve activity values for successive 350 epochs of a subject.

FIGS. 5 and 6 illustrate the mean R-R intervals and aSKNA values from the study mentioned above with respect to FIG. 4.

FIGS. 7A and 7B illustrate boxplot representation of HRV parameters in FIG. 4, and the SKNA parameters shown in FIG. 5.

FIG. 8 provides a table of performance of sleep stages classification models on a validation set using a set of 25 features, and a reduced set from those 25 features, for the study mentioned above with respect to FIG. 4.

FIG. 9 illustrates how the reliability parameter Cohen's kappa changes with adding features starting at the most relevant one for fourteen (14) heart rate variability (HRV) and eleven (11) SKNA features extracted from the study mentioned above with respect to FIG. 4.

FIGS. 10A-10C illustrate the transformed representation of a confusion matrix for 5, 3, and 2 sleep stages recognition, respectively.

FIG. 10D illustrates performance measures for 2 stages sleep classification.

FIG. 11 illustrates preprocessing steps for features extraction.

### DETAILED DESCRIPTION OF THE ILLUSTRATIVE EMBODIMENTS

The following Detailed Description refers to the accompanying drawings that illustrate exemplary embodiments. Other embodiments are possible, and modifications can be made to the embodiments within the spirit and scope of this description. Those skilled in the art with access to the teachings provided herein will recognize additional modifications, applications, and embodiments within the scope thereof and additional fields in which embodiments would be of significant utility. Therefore, the Detailed Description is not meant to limit the embodiments described below.

In the Detailed Description herein, references to “one embodiment,” an “embodiment,” and “example embodiment,” etc., indicate that the embodiment described may include a particular feature, structure, or characteristic, by every embodiment may not necessarily include the particular feature, structure, or characteristic. Moreover, such phrases are not necessarily referring to the same embodiment. Further, when a particular feature, structure, or characteristic may be described in connection with an embodiment, it may be submitted that it may be within the knowledge of one skilled in art to affect such feature, structure, or characteristic in connection with other embodiments whether or not explicitly described.

Embodiments of the subject disclosure provide a system that can include a wearable monitor having one or more sensors, and, moreover, one or more multi-modal, wearable sensor technologies, that are configured to capture, including sense or measure, information from a subject wearing the wearable sensor body that is used by the system to characterize different stages of sleep of the subject. According to certain embodiments, the sensor(s) of the wearable monitor are configured to contact an outer surface of a skin of a sleeping subject and sense, detect, measure, or otherwise obtain (collectively referred to herein as capture) information regarding at least a skin nerve activity (SKNA) and/or a heart rate of the subject. The system can further include a controller having, or communicatively coupled to, an optimization module or circuitry and/or an artificial intelligence engine having a support vector machine, that can derive, from information captured by the sensor(s), a plurality of activity features relating to the sleep of the subject. Moreover, the artificial intelligence engine can utilize machine learning to automate the process of characterizing, based at least in part on the activity features extracted from the captured information, the different stages of sleep of the subject. Such characterization of the different stages of sleep can be based at least in part on identification of one or more unique nerve activity signatures and/or heart rate variability signatures, as extracted from the captured information, for the different stages of sleep. For example, the system can include one or more, if not a plurality, of machine learning models that can use time domain and frequency domain features from the captured information, and, more specifically, nerve activity and heart rate variability information captured by the one or more sensors, as a subject transitions into different stages of sleep, to classify, including identify, the subject's different stages of sleep.

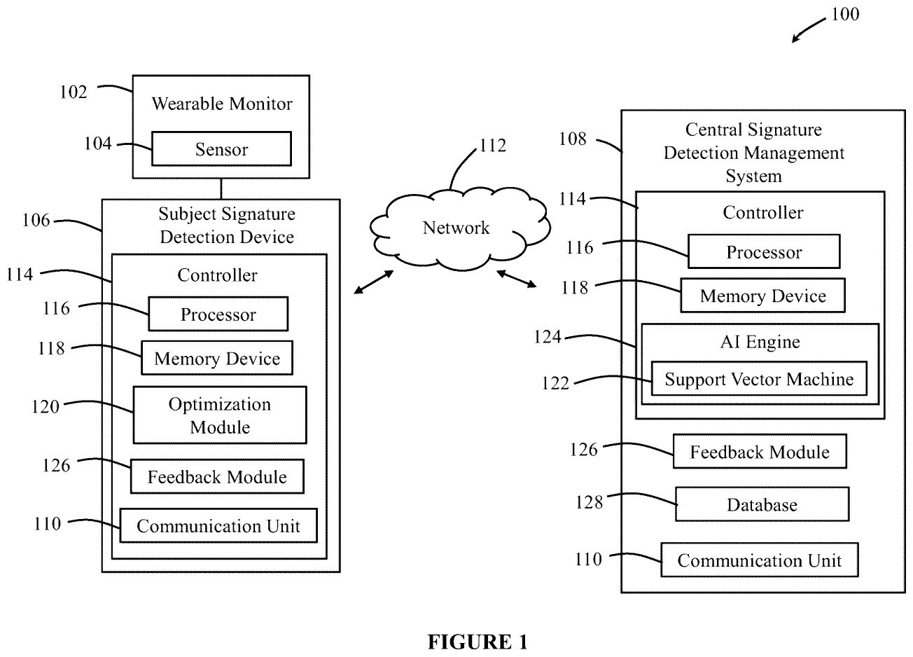

FIG. 1 illustrates a simplified block diagram of a system 100 for characterizing different stages of sleep utilizing information captured from the skin of a subject. The system 100 includes at least one wearable monitor 102 that is configured to be secured to the subject. Moreover, the wearable monitor 102 can be configured in a variety of manners to secure the wearable monitor 102 to the subject. For example, according to certain embodiments, the wearable monitor 102 comprises a body portion, such as, for example, a substrate, that can include, or be coupled, to an adhesive layer. The adhesive layer can comprise an adhesive that, when in contact with an outer surface of the skin of the patient, adheres to the outer surface of the skin of the patient, thereby securing, attaching, or affixing the wearable monitor 102 to the subject. Alternatively, adhesive strips can extend over, or from, a portion of the body portion of the wearable monitor 102 and to positions at which the adhesive strips can be secured to the skin of the subject. However, the wearable monitor 102 can be secured to the subject in a variety of other manners, including but not limited to, use of one or more straps that can be attached, removably coupled to, or part of a monolithic structure of the body portion that can extend around, including enclose, at least a portion of the subject. According to certain embodiments, one or more attachment members, including but not limited to hook and loop material, buttons, or snaps, among others, can be utilized to secure a position of the strap(s) and/or body portion such that the wearable monitor 102 can be selectively secured around portions of the subject. Alternatively, or additionally, according to certain embodiments, the wearable monitor 102 can comprise a portion of an article of clothing, including, for example, a smart shirt, or an accessory, such as, for example, a smart watch, bracelet, or ring, that is configured to secure at least a portion of the wearable monitor 102 to the skin of the subject.

The wearable monitor 102 can further include one or more sensors 104. According to certain embodiments, at least one sensor 104 is configured to capture information regarding skin nerve activity (SKNA) of the subject, while another sensor 104 is configured to capture information regarding a heart rate of the subject. According to other embodiments, a single sensor 104 can be configured to capture, including measure, information used to determine both the skin nerve activity and the heart rate of the subject. For example, according to certain embodiments, the sensor 104 can comprise at least one, if not a plurality, of electrocardiogram (ECG) patch electrode sensors or new ECG devices that can simultaneously sense both ECG and SKNA signals.

The system 100 can further include a subject signature detection device 106 that is communicatively coupled to at least the sensor(s) 104 of the wearable monitor 102, including via a wired or wireless connection. For example, according to certain embodiments, the subject signature detection device 106 can be a smartphone, tablet, laptop, or personal computer, among other computing devices. According to such embodiments, the subject signature detection device 106 can wirelessly communicate with the wearable monitor 102, including, for example, via Bluetooth, Bluetooth Low Energy (BLE), and near-field communications (NFC), among other wireless communication protocols. Additionally, according to certain embodiments, the subject signature detection device 106 and the wearable monitor 102 can be incorporated into the same device, including, for example, a smartwatch or smart shirt, among other wearable smart devices.

The system 100 can further include a central signature detection management system 110 that can be remote from the subject signature detection device 106. Thus, for example, the subject signature detection device 106 and the central signature detection management system 110 can each include, or be communicatively coupled to, a communication unit 110. The communication units 110 can be used in communications between the subject signature detection device 106 and the central signature detection management system 110 over a network 112, including, for example, over a wireless network, such as, for example, internet, cellular, and/or Wi-Fi networks, as well as combinations thereof, among others.

The central signature detection management system 110 can be configured to receive captured information and/or determinations made by one or more, if not a plurality, of subject signature detection devices 106 of one or more subjects. As discussed below, information received by the central signature detection management system 110 from different subjects can be utilized by a support vector machine 122 to improve, via one or more machine learning techniques, one or more models utilized by the central signature detection management system 110 and/or the subject signature detection device 106 for identifying different stages of sleep of a subject.

The subject signature detection device 106 and the central signature detection management system 110 can each include one or more controllers 114 having at least one processor 116 and at least one memory device 118. The controller 114, processor(s) 116, and/or memory device(s) 118 can, or cannot, be dedicated to the operation of the system 100. Thus, for example, with respect to the subject signature detection device 106, according to certain embodiments, the processor 116 can comprise one or more processors, including compute circuits, that can be utilized to control operation of the subject signature detection device 106, and, optionally, can also be utilized in connection with controlling one or more other operations or components of the system 100. Therefore, according to certain embodiments, one controller 114, including one or more processors 116 of that controller 114, can be utilized to control operation of at least the subject signature detection device 106, or the corresponding components, portions, or segments of the subject signature detection device 106. Alternatively, a plurality of controllers 114, or combinations of processors 116, including compute circuits, can be utilized to control operation of the subject signature detection device 106, as well as control operations of different components or systems of the system 100. Thus, for example, while certain embodiments herein may mention functions being performed by a controller 114, including the associated processor 116, such functions can be performed by a single controller or processor, or, alternatively, one or more functions can be performed by one or more controllers or processors, and one or more other functions can be performed by one or more other controllers or processors or combinations of controllers or processors.

The memory device 118 can have instructions stored therein that are executable by the processor 116 to cause the processor 116 to receive input from one or more sensors 104 of the wearable monitor 102, among other portions of the subject signature detection device 106. The processor 116 can be embodied as, or otherwise include any type of processor, controller, or other compute circuit capable of performing various tasks such as compute functions and/or controlling the functions of at least the subject signature detection device 106. For example, the processor 116 can be embodied as a single or multi-core processor(s), a microcontroller, or other processor or processing/controlling circuit. In some embodiments, the processor 116 can be embodied as, include, or otherwise be coupled to an FPGA, an application-specific integrated circuit (ASIC), reconfigurable hardware or hardware circuitry, or other specialized hardware to facilitate performance of the functions described herein. Additionally, the processor 116 can be embodied as, or otherwise include a high-power processor, an accelerator co-processor, or a storage controller.

The memory device 118 can be embodied as any type of volatile (e.g., dynamic random-access memory (DRAM), etc.) or non-volatile memory capable of storing information therein. Volatile memory may be embodied as a storage medium that requires power to maintain the state of information stored by the medium. Non-limiting examples of volatile memory may include various types of random-access memory (RAM), such as dynamic random-access memory (DRAM) or static random-access memory (SRAM). One particular type of DRAM that may be used in a memory module is synchronous dynamic random-access memory (SDRAM).

In some embodiments, the memory device 118 can be embodied as a block addressable memory, such as those based on NAND or NOR technologies. The memory device 118 can also include future generation nonvolatile devices, such as a three-dimensional crosspoint memory device (e.g., Intel 3D XPoint™ memory), or other byte addressable write-in-place nonvolatile memory devices. In some embodiments, the memory device 118 can be embodied as, or can otherwise include, chalcogenide glass, multi-threshold level NAND flash memory, NOR flash memory, single or multi-level Phase Change Memory (PCM), a resistive memory, nanowire memory, ferroelectric transistor random access memory (FeTRAM), anti-ferroelectric memory, magnetoresistive random access memory (MRAM) memory that incorporates memristor technology, resistive memory including the metal oxide base, the oxygen vacancy base and the conductive bridge Random Access Memory (CB-RAM), or spin transfer torque (STT)-MRAM, a spintronic magnetic junction memory based device, a magnetic tunneling junction (MTJ) based device, a DW (Domain Wall) and SOT (Spin Orbit Transfer) based device, a thyristor based memory device, or a combination of any of the above, or other memory. The memory device 118 can refer to the die itself and/or to a packaged memory product. In some embodiments, 3D crosspoint memory (e.g., Intel 3D XPoint™ memory) can comprise a transistor-less stackable cross point architecture in which memory cells sit at the intersection of word lines and bit lines and are individually addressable and in which bit storage is based on a change in bulk resistance.

The controller 114 of the subject signature detection device 106 and/or the central signature detection management system 110 can also include a machine learning optimization module or circuitry 120 that can be utilized to identify, from certain nerve activity signatures and/or heart rate variability signatures, a particular sleep stage of the subject, as further discussed below. Such signatures can be derived, and updated, via use of at least the optimization module 120 based on one or more models, including algorithms, and inputted information that can include information provided by, or derived from, the sensor(s) 104, the subject, and/or an operator of the system 100, among other input information. According to certain embodiments, such machine learning for either or both the development or refinement of the model(s), including algorithms, used by the optimization module 120 can be performed online at the signature detection device 106, or offline, including, for example, at the central signature detection management system 110 via training of a support vector machine 122 of an artificial intelligence (AI) engine 124 at the central signature detection management system 110.

The system 100 can further include a feedback module or circuitry 126 that can be located at either or both the system 100 and the central system 100. The feedback module 126 can include a recording of information that can be used by the optimization module 120 and/or support vector machine 122 to improve the accuracy of the associated machine learning models. For example, the feedback module 126 can include observations, inputs, and or adjustments made by the subject or other operator of the system 100 with respect to prior determinations made by the model(s) of the optimization module 120 and/or neural network 122 relating to prior characterizations relating to identified sleep stages. The optimization module 120 and/or neural network 122 can use such feedback from the feedback module 126, including, for example, based on comparisons of such feedback with prior determinations, including predictions, attained from the machine learnable model. Moreover, such feedback can assist in identifying, or further refining identified, patterns or other trends, among other machine learning techniques, that can be used by the optimization module 120 and/or neural network 122 to improve the models used by the system 100 to identify different stages of sleep for the subject based on at least information provided by the sensor 104. For example, such machine learning can include improving the models and/or weighted values used by the models, among other modifications relating the model(s) 100 used by the system, 100 in characterizing different sleep stages using at least the captured information provided by the one or more sensors 104.

According to the illustrated embodiment, information captured by the sensor 104 can be used by the controller 114 to identify variances in skin sympathetic nerve activity that can occur between sleep stages. Such captured information can also be simultaneously recorded, such as, for example, by the memory device 118. Such sympathetic nerve activities are typically composed of muscle sympathetic nerve activity (MSNA) and skin sympathetic nerve activity (SKNA). With respect to MSNA, detection of changes or variances, as captured by the sensor 104, in MSNA can correspond to different sleep stages. With respect to SKNA, captured information obtained by the sensor 104 relating to sympathetic nerve activity from the skin can also provide, or be used to derive, information relating to one or more SKNA related activity features that vary for different stages of sleep. Such SKNA related activity parameters can include, for example, an aSKNA parameter, which can indicate the average of nerve activity per sample, or burst frequency, which can differ between sleep stages.

The central signature detection management system 108 and/or subject signature detection device 106 can further include a database 128 that can contain a plurality of information relating to identified or historical ECG and SKNA measurements, including associated historical activity features extracted from those historical measurements, and the corresponding identified sleeping stage. Such known, or pre-identified information can be used at least as reference information in connection with machine learning or training of one or more models of the optimization module 120 and/or the neural network 122 of the AI engine 124 for identifying different sleep stages based on activity features extracted from ECG and SKNA measurements. Such historical information can be utilized in connection with not only developing the associated machine learnable model(s) for identifying different sleep stages based on ECG and/or SKNA measurements, but also, over time, further refining the model(s) via subsequent machine learning. Further, according to certain embodiments, information provided by the database 128 can include information attained from the above-discussed feedback module(s) 126.

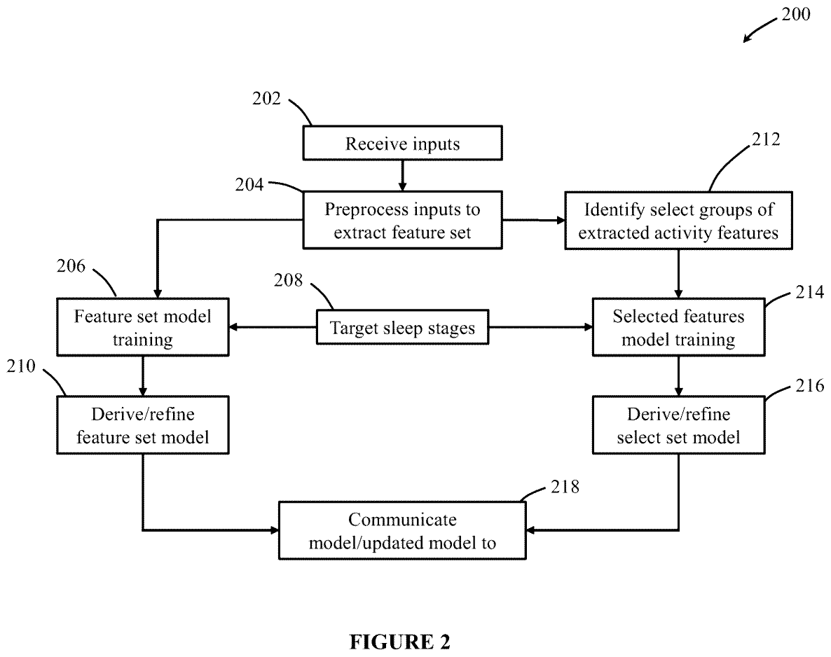

FIG. 2 illustrates an exemplary method 200 for training and developing first and second models based on an activity feature set, and a selected subset of the activity features, respectively derived from ECG and SKNA measurements for characterizing different stages of sleep of a subject. The method 200 is described below in the context of being carried out by the illustrated exemplary system 100. However, it should be appreciated that method 200 can likewise be carried out by any of the other described implementations, as well as variations thereof. Further, the method 200 corresponds to, or is otherwise associated with, performance of the blocks described below in the illustrative sequence of FIG. 2. It should be appreciated, however, that the method 200 can be performed in one or more sequences different from the illustrative sequence. Additionally, one or more of the blocks mentioned below may not be performed, and the method 200 can include steps or processes other than those discussed below.

At block 202, inputs can be received by a controller 114, including, for example, the controller 114 of one or more of the subject signature detection device 106 or central signature detection management system 108. The inputs received at block 202 can be provided by the one or more sensors 104 of the wearable monitor 102, and moreover, can correspond to a plurality of captured information corresponding to ECG and SKNA measurements obtained from the sensor(s) 104 of the wearable monitor 102 as the subject wearing the wearable monitor 102 is sleeping.

At block 204, an activity features set can be extracted from the inputted information received that block 202. The activity features set can include a first group of extracted activity features and a second group of extracted activity features. The first group of extracted activity features can correspond to one or more features extracted from SKNA measurements provided by block 202, while the second group of extracted activity features can correspond to heart rate variability related features that are extracted from the ECG measurements that were provided at block 202. Additionally, the activity features set, and associated first and second group of extracted activity features, can correspond to different intervals, such as, for example, time-based intervals and/or intervals relating to a predetermined series length and predetermined number of epochs, among other types of intervals. For example, a first activity features set can correspond to features derived from the SKNA and ECG measurements during a first interval, such as, for example, over a 5-minute duration, and a second activity features set can correspond to another, or subsequent, 5-minute duration. In such an embodiment, the first and second group of extracted activity features utilized for the first and second activity features sets may be of the same type (e.g., aSKNA, varSKNA, MeanNN, SDNN, etc.), but the corresponding values of the first and second group of extracted activity features for the first activity features set may, or may not be, different than the corresponding values obtained for the second activity features set. In such situations, differences, if any, in the values for the first and second activity features sets between the first and second activity features sets may be dues to the first and second activity features sets corresponding to different sleep stages.

The number and types of features for the first and second groups of extracted activity features for the activity features set can be based on a variety of criteria. According to certain embodiments, the number and types of features for the first and second activity features sets of the activity features set can correspond to extracting from the inputted information received at block 202 as many activity features as possible for each of the first and second activity features that may have some relevancy to the model identifying an associated stage of sleep. In this regard, the activity features set can be referred to as a full set of activity features.

With this approach, machine learning can be used at block 206 to derive a first model, also referred to as a feature set model, for identifying sleep stages using the full set of activity features. Again, with such an approach, the model can be at least initially developed utilizing historical information, including, for example information provided by the database 128, as generally indicated by block 208. Moreover, the information provided by block 208 can correspond to known, or preidentified, information that associates the types of information provided by the first and second features and a particular sleep stage. Thus, for example, the information provided by at least block 208 can be used by the machine learning first model to identifying patterns with respect to the first and second features, as well as combinations thereof, and the associated known sleep stage. Such identified patterns can then be further refined by the information provided from the feature set drive at block 204. Additionally, such training of the model can occur, for example, at either, or both, the optimization module 120 and/or the support vector machine 122 of the AI engine 124. Further, as additional information is gathered, such as, for example, via use of a feedback module 126, such information can also be provided at block 206 to further refine the first, or feature set, model. The development of the first, full set model, and any associated update or refinement of that model can be outputted at block 210.

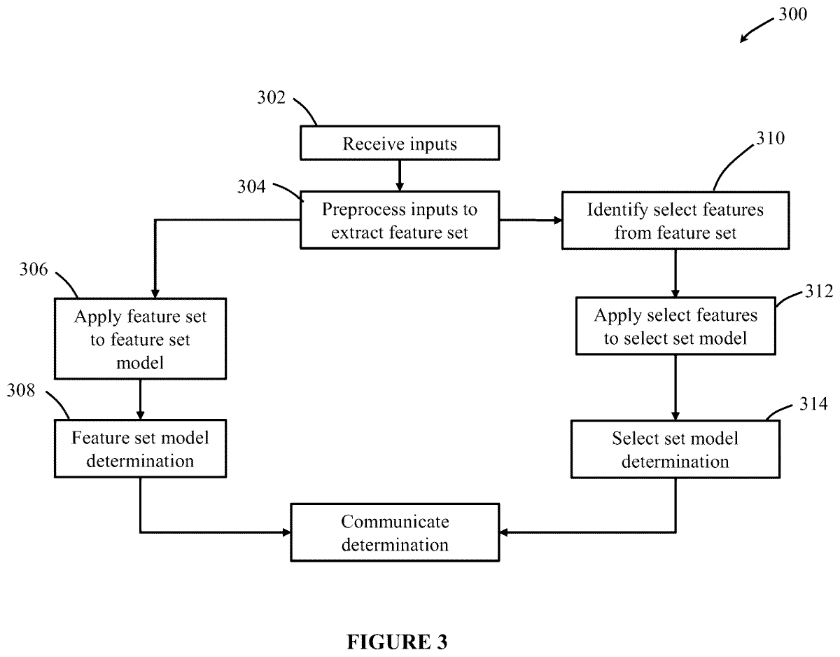

The process of providing information for model training at block 206 from either or both blocks 204 and 208 to block 206 can be conducted regularly, or semi-regularly, in an attempt to continuously improve the accuracy of the first model. Thus, the first model provided at block 210 can undergo changes or adjustments as the model is improved by other training that occurs at block 206. The derived model from block 210 can subsequently be used in connection with categorizing, including determining, different sleep stages of a subject wearing the wearable monitor 100, as discussed, for example, below with respect to FIG. 3.

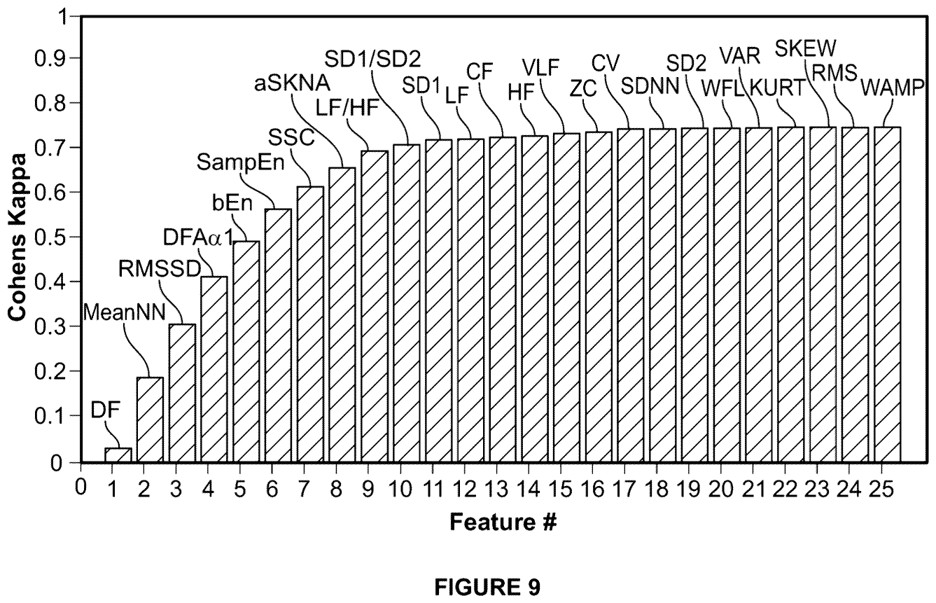

However, as discussed below with respect to EXAMPLE I, and particularly in connection with FIG. 9, certain activity features extracted from SKNA and ECG measurements may be more indicative than other activity features as to the associated sleep stage of the subject. Further, a reduction in the number of activity features for either or both the first and second groups of extracted activity features, and thus reduce the number of variables inputted into the machine learning model, can reduce computational cost and, in some cases, can improve classification performance. The below discussion regarding “EXAMPLE I” provides examples of 11 select SKNA signals that can provide the SKNA features for a selective first group of extracted activity features (also referred to as a first select group of extracted activity features), and examples of 10 HRV parameters and 4 frequency domain parameters that can be utilized for a selective second group of extracted activity features (also referred to as a second select group of extracted activity features). However, according to other embodiments, different features in addition, or in lieu, of those discussed below with respect to EXAMPLE I, as well as different combinations of features can be utilized for either, or both, the above-discussed first and second select groups of activity features. Further, such identified features can vary over time as the machine learnable model is refined or improved with respect to the accuracies of the determinations outputted from the model.

Thus, as an alternative to the above-discussed first model, and moreover use of the full set of activity features, according to certain embodiments, the first and second select groups of extracted activity features from block 202 can be selected or extracted at block 212. According to certain embodiments, the first and second select groups of activity features may comprise one or more subsets of the full set of extracted activities. Alternatively, rather than extracting or selecting each activity feature associated with a full set of activity features at block 212, a more selective approach can be taken to identifying which activity features are to be extracted from the information inputted at block 202 in deriving either, or both, the first and second groups of extracted activity features. Such a selective approach to extracting activity features from the inputted information can be based on a variety of criteria. For example, as discussed below, which activity features are to be utilized for either, or both, the first and second groups of extracted activity features can, at least in part, be based on experimental analysis. Additionally, or alternatively, the identification of the activity features for either, or both, the first and second groups of extracted activity features can be the product of machine learning, including, for example, machine learning that identifies which activity features, or combinations of activity features, provide an accurate representation of the corresponding sleep stage actually being experienced by the subject. Thus, the optimization module 120 and/or support vector machine 122 may be engaged in a machine learning process in which historical information, including, but not limited to, reference and/or feedback information is evaluated to identify patterns that may assist in the model identifying which activity features, or combination of activity features, are, or are not, to be utilized in determining, including predicting, the associated sleep stage of the subject.

Thus, unlike the first, feature set model derived or trained at block 206 that utilized the full set of activity features, at block 214, a second, select set model can be derived and trained using a select set of activity features that can comprise select activity features, as identified by the first and second select groups of activity features. Aside from being based on the use of select, rather than full, sets of activity features, the development and training of the second, select set model at block 214 can be similar to that discussed above with respect to the first, feature set model at block 206. However, as the second, select set model utilizes fewer activity features than the first, feature set model, the development of the second, select set model may be more robust and utilize less computational power, which can, in at least certain instances, enhance the accuracy or at least provide the same accuracy of the first, feature set model (206). The development of the second, select set model, and any associated update or refinement of that model can be outputted at block 216.

According to certain embodiments, deriving or determining one, if not both, of the first, full set model and the second, select set model can occur at the central signature detection management system 108 utilizing the support vector machine 122. According to such an embodiment, the model being developed or adjusted/updated by the central signature detection management system and/or being used by the subject signature detection device 106 can be communicated to the subject signature detection device 106 at block 218. In certain instances, the model can be operated on the subject signature detection device 106 as, or as part of, a software application (“app.”), such as, for example, an app. on a smartphone, among other computing devices.

The process of providing information for model training at block 214 from either or both blocks 208 and 212 to block 214 can be conducted regularly, or semi-regularly, in an attempt to continuously improve the accuracy of the second, select set model. Thus, the second model provided at block 216 can undergo changes or adjustments as the model is improved by other training that occurs at block 214. The derived model from block 216 can subsequently be used in connection with categorizing, including determining, different sleep stages of a subject wearing the wearable monitor 100, as discussed, for example, below with respect to FIG. 3.

FIG. 3 illustrates an exemplary method 300 for utilizing either, or both, the first model and second model identified in FIG. 2 to generate a determination of stages of sleep of the subject. The method 300 is described below in the context of being carried out by the illustrated exemplary system 100. However, it should be appreciated that method 300 can likewise be carried out by any of the other described implementations, as well as variations thereof. Further, the method 300 corresponds to, or is otherwise associated with, performance of the blocks described below in the illustrative sequence of FIG. 3. It should be appreciated, however, that the method 300 can be performed in one or more sequences different from the illustrative sequence. Additionally, one or more of the blocks mentioned below may not be performed, and the method 300 can include steps or processes other than those discussed below.

At block 302, information similar to that discussed above with respect to block 202 can be received by the controller 114 of either, or both, the subject signature detection device 106 and the central signature detection management system 108. Moreover, the information provided at block 302 can at least include captured information obtained by the sensor(s) 104 of the wearable monitor 102. Again, such information can include SKNA and ECG information measured over a duration, or different periods, of time.

At block 304, features from the inputted information received at block 302 can be processed to extract features for the first and second groups of activity features, as discussed above with respect to at least block 204. Alternatively, at block 310, at block 304, the first and second select groups of activity features can also be obtained. Accordingly, at block 306, the extracted first and second groups of activity features from block 304 can be applied to the first model, also referred to as the feature set model, and a determination, including a prediction, of the stage of sleep corresponding to the provided feature set can be outputted by the first model at block 308. Alternatively, at block 312, the first and second select groups of activity features can be applied to the second model, also referred to as the select set model, and the select set model can output a determination, including a prediction, of the stage of sleep corresponding to the provided select set of features.

The outputs provided by one or both of the first and second models at blocks 308 and 314 can be communicated to the subject, among others, in a variety of different manners. For example, according to certain embodiments, the controller 114 of the subject signature detection device 106 and/or the central signature detection management system 108 can generate one or more signals to communicate the determination made at block 308 and/or block 314 via an output device of the subject signature detection device 106 and/or the central signature detection management system 108, such as, for example, a monitor, display, touch screen, and/or speaker. Additionally, the determinations made at either, or both, block 308 and block 314 can be recorded, such as, for example, stored by the memory device 118 selective review at the convenience of the subject, among others. Further, as previously mentioned, according to certain embodiments, the first and/or second model can be operated on the subject signature detection device 106 as, or as part of, a software application (“app.”), such as, for example, an app. on a smartphone, among other computing devices in which event the output of the associated first or second model(s) can be outputted via an associated output device, including, for example, a display, touchscreen, monitor, and/or speaker, among other output devices.

### Example I

In this study, the sleep stages were classified using features derived from the ECG and SKNA. The study was performed on 21 subjects, and 14 heart rate variability (HRV) and 11 SKNA features for a total of 25 features were initially extracted. From those 25 features, the 17 most relevant (12 HRV, 5 SKNA) features were selected. Both individual and combined performance of HRV and SKNA were evaluated for classification of 5 basic sleep stages, 3 (NREM, REM, WAKE), and binary (SLEEP vs WAKE) stages. The study showed that a machine learning algorithm that uses features from SKNA and HRV has an average recognition accuracy of 92.02%, 95.59%, and 96.33% for the recognition of 5, 3 and binary sleep stages on 10-fold cross validation set. This AI-powered sleep classification system provides an advancement in real-time sleep monitoring.

The subjects for the study were between the ages of 28 to 64 years, consisting of 12 males and 8 females, with 12 of the subjects being white and the other 8 being black or others, and having body mass indexes of 24.2 to 36.7. The ECG and SKNA signals were recorded from a study population of 21 patients suffering from obstructive sleep apnea syndrome, and they were subject to continuous positive airway pressure (CPAP) treatment. The ECG and SKNA signals were recorded simultaneously from the chest using the newECG device at the sampling rate of 10 KHz. The ECG signal and SKNA was extracted from the signal by applying bandpass filters of [0.05 41] Hz and [500 1000] Hz, respectively. The R-R series was extracted from the ECG using Labchart Pro 2008.

A total of 17,549 epochs were analyzed from sleep studies that last on average 7 hours. There were 1,864, 7,337, 2,013, 2,555, 3,780 epochs for NREM-1, NREM-2, NREM-3, REM, and WAKE stages respectively, which corresponded to 10.62%, 39.55%, 11.47%, 14.56%, and 21.54% of the total epochs The combination of information from HRV and SKNA improves the sleep stages recognition obtained using either HRV or SKNA only. The results found in this study contribute to the deeper understanding of SKNA for sleep study and open the door for sleep monitoring using a single wearable monitor.

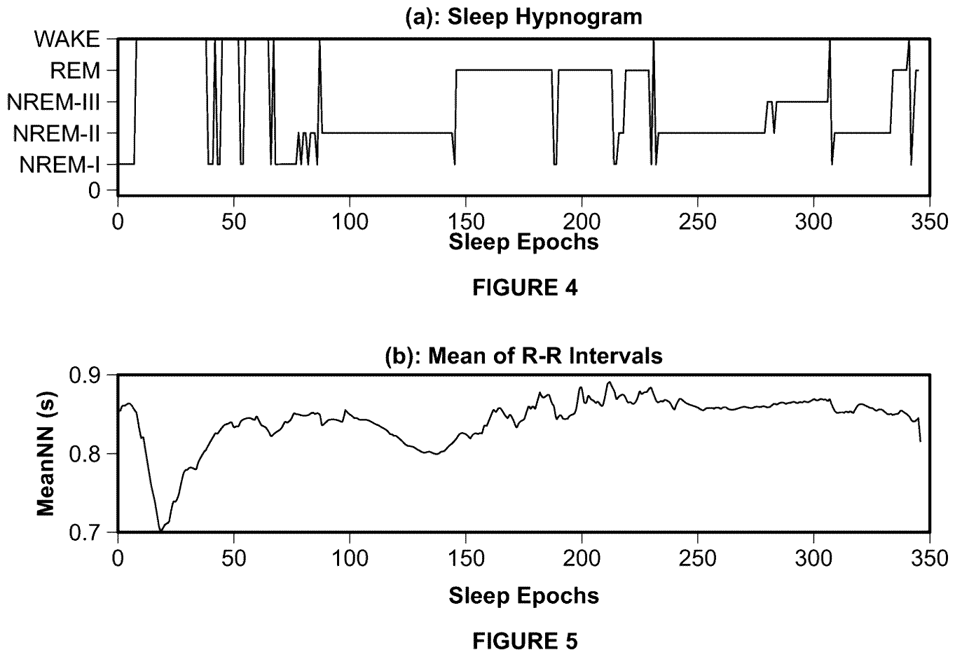

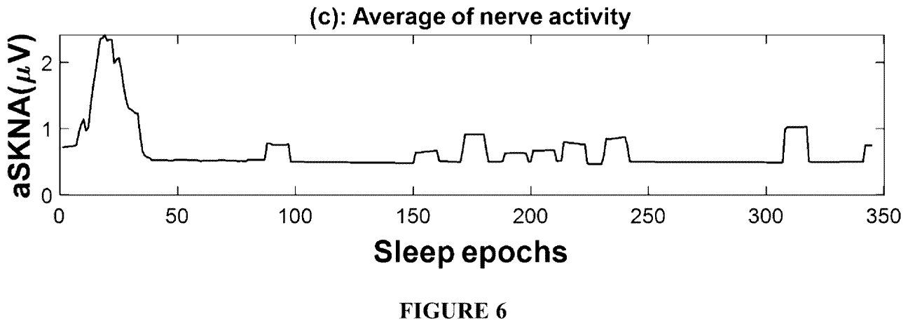

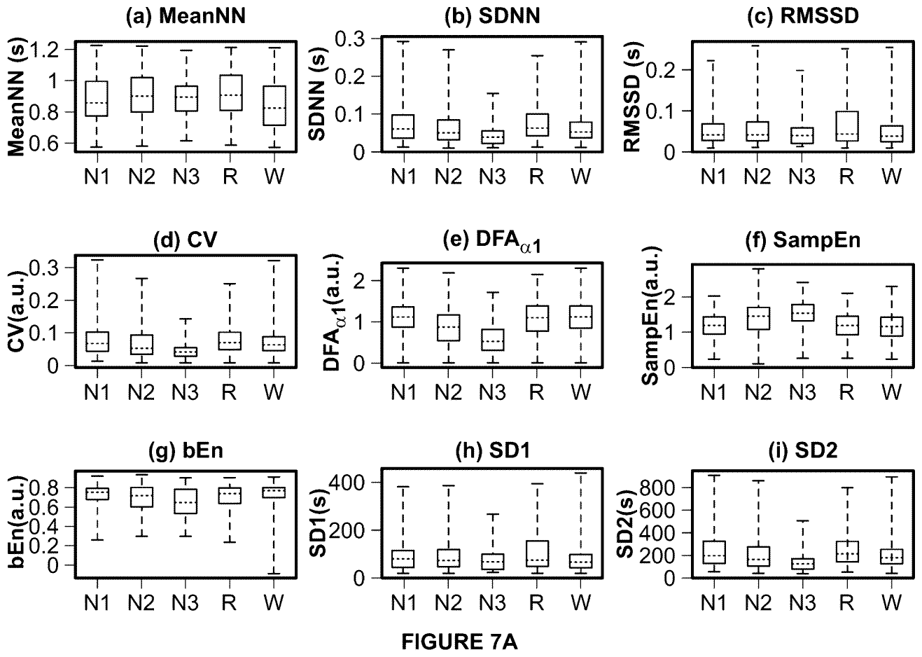

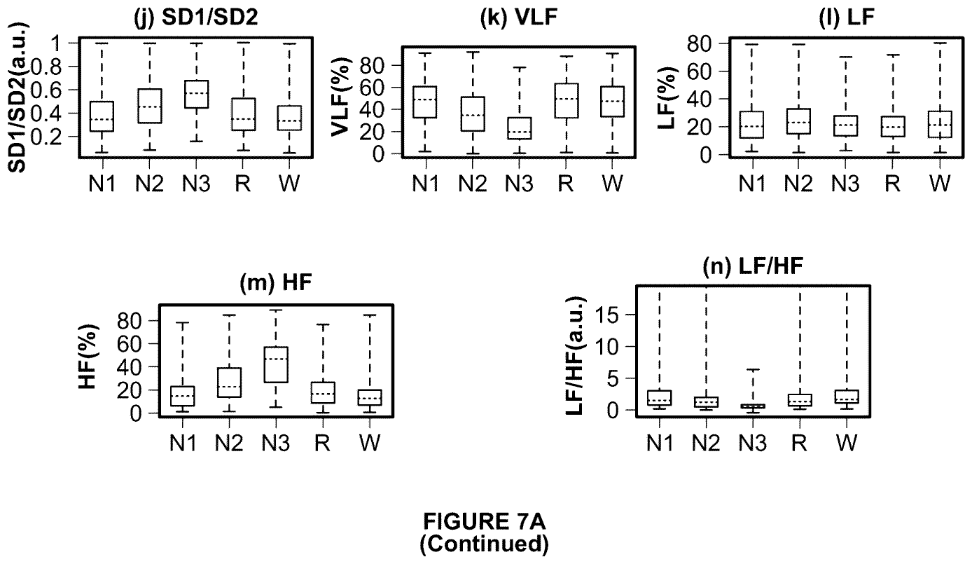

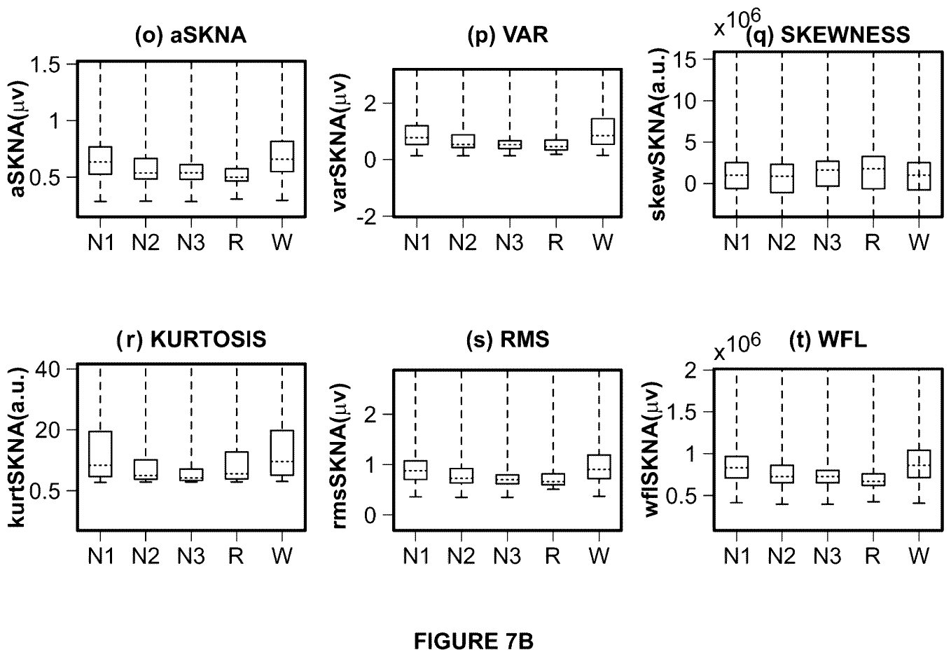

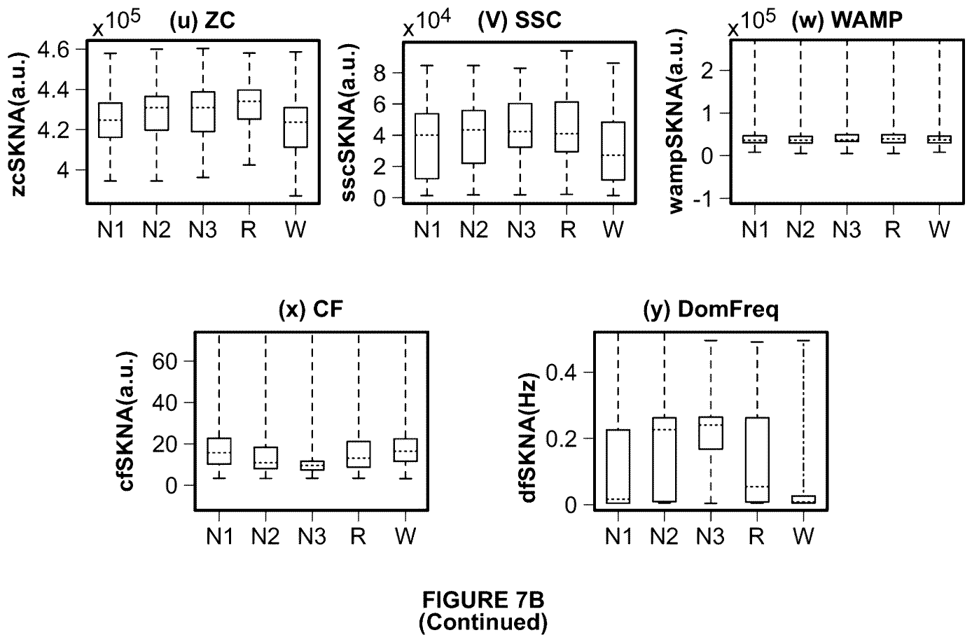

FIG. 4 illustrates the sleep hypnogram derived from a study using mean values of the R-R intervals and skin sympathetic nerve activity values for successive 350 epochs of a subject. FIGS. 5 and 6 illustrate the mean R-R intervals and aSKNA values from the study mentioned above with respect to FIG. 4. From Figures X-Z, both the heart rate and skin sympathetic nerve activity increases during WAKE and REM compared to NREM. Heart rate variability (HRV) parameters in different sleep stages are represented by the block diagrams in FIGS. 7A and 7B.

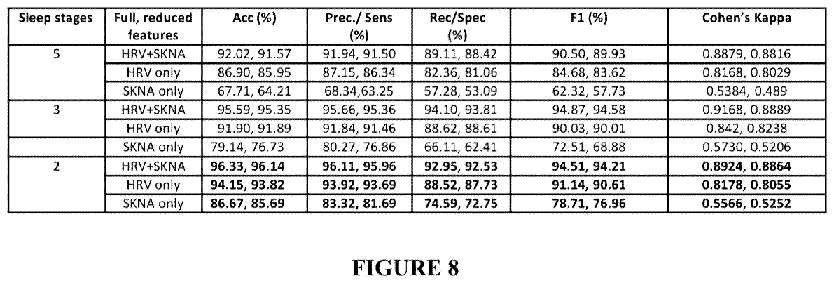

The classification performance for five sleep stages using all features from HRV and SKNA is provided in the table shown in FIG. 8. Moreover, the table in FIG. 8 indicates the performance of sleep stage classification models on a validation set using the set of all features and a reduced set (selected). Here, the values of performance metrics represent the average values of 10-folds.

The set of 25 features (14 from HRV and 11 from the SKNA analysis), and a subset of 12 HRV and 5 SKNA features were selected as the most relevant by the greedy forward approach based on Cohen's Kappa to distinguish sleep stages. The selected features and the corresponding Cohen's kappa are shown in FIG. 9. In FIG. 9, the order of important features and the corresponding Cohen's kappa are displayed. SKNA features were identified as DF, SSC, aSKNA, CF, ZC, WFL, VAR, KURT, SKEW, RMS, and WAMP. Additionally, the left most feature corresponds to the most important, with the features ranked from left to right or most important to less important.

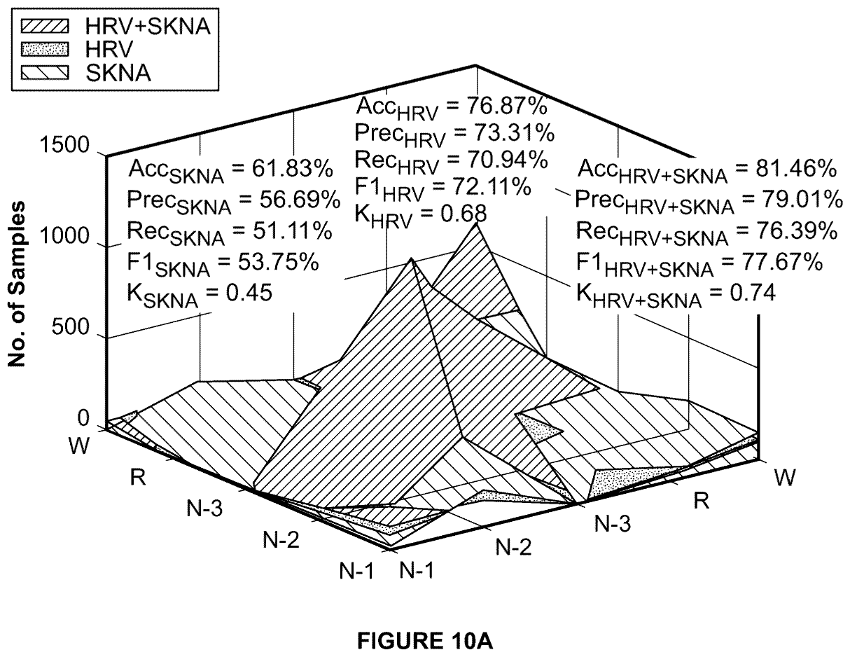

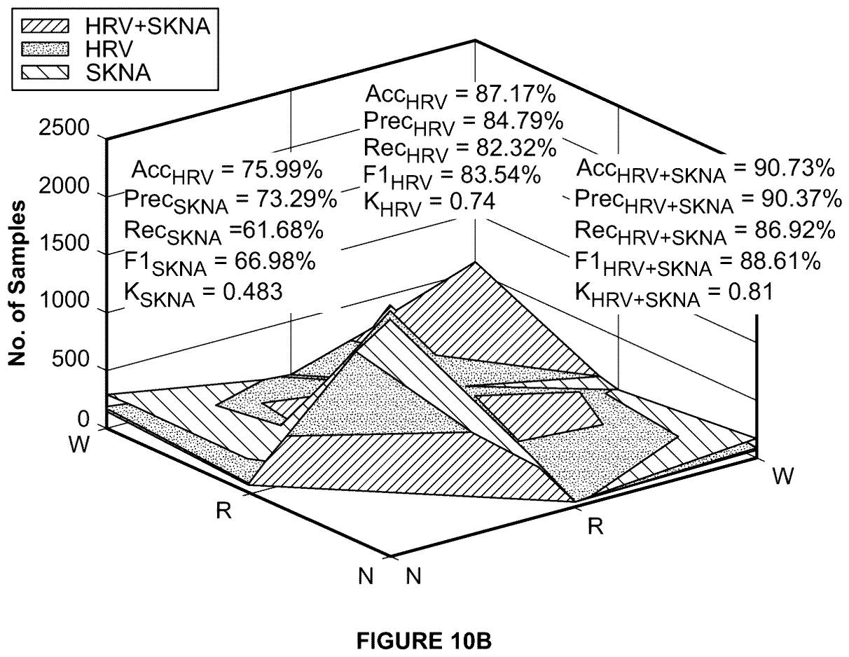

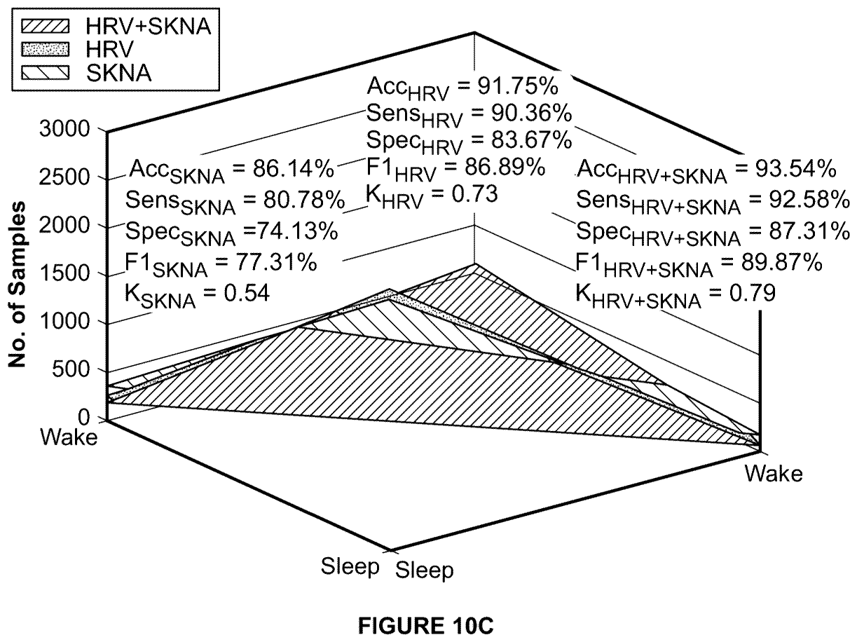

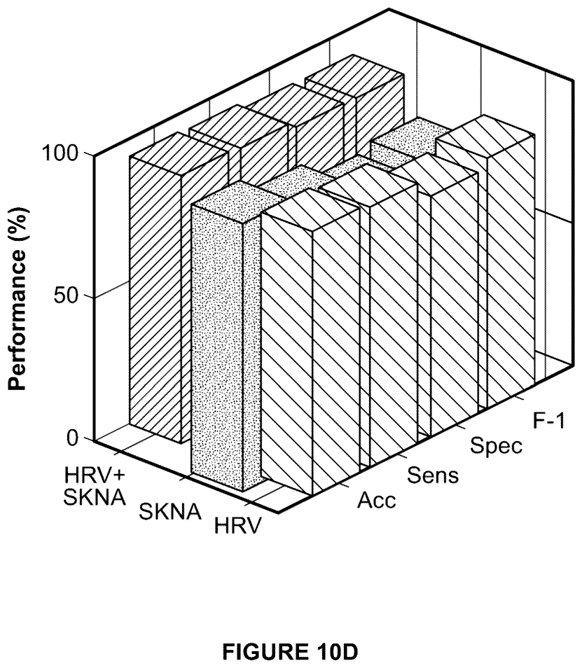

As seen from FIG. 9, the dominant frequency (DF) of the SKNA signal was found to be the most important, with the coefficient of variance (CV) identified as the lowest important feature of the relevant features set. The addition of other features after CV does not increase the Cohen's kappa. The performance metrics using the best relevant features remain almost the same to the performance obtained using the full set of features. The values of Accuracy (ACC), Precision/Sensitivity (Prec/Sens), Recall/Specificity (Rec/Spec), F−1 score (F1), and Cohen's kappa using the selected features were 91.574%, 91.49%, 88.42%, 89.93%, and 0.7716, respectively, for five-stage sleep classification. However, the values of performance measures are slightly reduced when evaluated on the separate test set. The confusion matrix presented by the Mesh-Grid for all classification problems using the best set of features on the separate test set are shown in FIGS. 10A-10D, FIGS. 10A-10C illustrate the transformed representation of the confusion matrix for 5, 3, and 2 sleep stage recognition, respectively, while FIG. 10D illustrates performance measures for 2 stages sleep classification. In these Figures, N, R, and W represents, NREM, REM, and WAKE, respectively.

The p-values of statistical tests (Wilcoxon RankSum or Student's t-test) were found significant (p<0.05) for these selected features to distinguish between SLEEP and WAKE, and most of them except RMSSD, SD1, and VLF [%] were also found significant to discriminate NREM, REM, and WAKE sleep stages. The p-value of McNemar was <<0.05 to discriminate between the predicted performance of the classification models obtained for HRV only versus the combination of HRV and SKNA parameters.

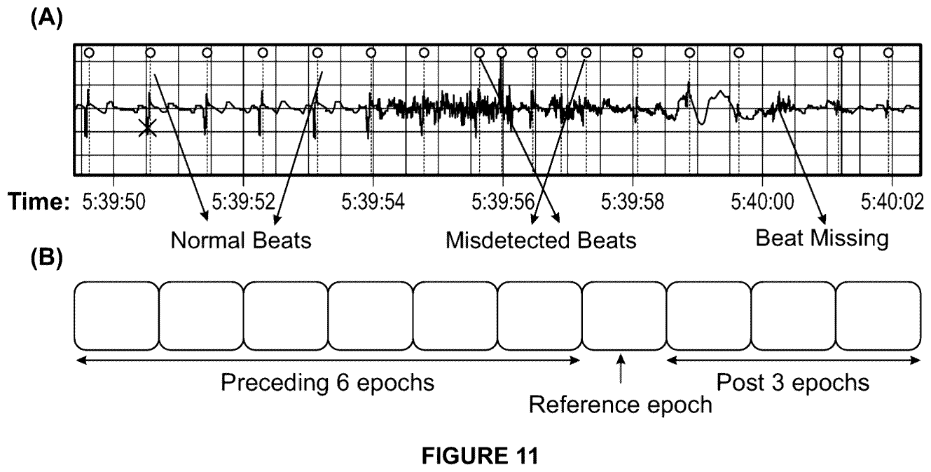

Any motion artifacts or extreme 60 Hz frequencies in the recordings were discarded. Additionally, the R-R series contained some values due to misdetection or missing a beat (FIG. 11). In one example, the study considered only R-R intervals which lies within 95% of its confidence interval. The series length, number of previous and post epochs might affect the classification accuracy. Since the main target was to investigate the feasibility of SKNA and its combination with HRV for sleep stage recognition, the deep study was not performed on the selection of epochs, but instead the series length and number of preceding and post epochs was followed using a single machine learning approach (SVM). Skin sympathetic nerve activity like the HRV parameters differ between sleep stages, and the study found more average nerve activity during WAKE than NREM and REM, which may be attributed to population for this study mostly coming from OSA patients who were subjected to the continuous positive airway pressure (CPAP), which can drastically reduce the SKNA value. The reduced mean R-R interval during WAKE means that the heart rate is increased during WAKE compared to SLEEP. As seen from FIGS. 7A and 7B, both HRV and SKNA parameters during sleep stages NREM-1 and WAKE are very similar, because NREM-1 is actually a transition between WAKE to SLEEP, which has led some researchers to propose the merging of NREM-1 with WAKE. However, the study kept it separate to follow the standard of manual sleep scoring. The features selection can be critical step in any classification problem, where the features are ranked based on a performance measure. A model can show better accuracy by chance with less important features than the robust ones. Thus, Cohen's kappa was considered instead of accuracy (Acc) as a standard for features selection. The study also identified the dominant frequency (DF) of SKNA as the most relevant feature out of all features from HRV and SKNA as depicted in FIG. 9 and previously identified. Furthermore, five other features of SKNA and 12 HRV features were identified as relevant, Although both SampEn and bEn measure the complexity of a time series, differences in computational methods can result in varying estimations based on the presence of nonstationary elements, upper and lower trends in the series. Consequently, the study proposed both entropy measures, and both were identified as important features. Additionally, the study proposed both linear, frequency, nonlinear, and regularity measures of HRV analysis, and nonlinear and regularity metrics, except for the mean and RMSSD, were selected as the most relevant features.

The study evaluated individual and the combined power of HRV and SKNA parameters for sleep stages classification from binary to 5 sleep stages using the full set of features and the reduced set of features, as seen by the table shown in FIG. 8. The best selected features provide similar performance with the full set of features. However, the value of recall has been slightly reduced with reduce set of SKNA only. This means the discarded features of SKNA improves recognition of WAKE when used SKNA only, but their absence is compensated by the HRV parameters when used in combination. The HRV provides better performance than the SKNA for the classification problems of 3 and 5 sleep stages as shown in FIG. 5. However, their performance is very comparable for binary (SLEEP vs WAKE) classification problems as shown FIG. 10D. Thus, when interested only in sleep quality rather than staging each phase, SKNA can be used for long-term sleep monitoring in noninvasive way. The combination of HRV and SKNA parameters significantly improves their individual performance for 3 and 5 sleep stages. This means that SKNA parameters captures some information to discriminate between the basic sleep stages that HRV parameters alone cannot and vice-versa.

The manual sleep scoring was done on different source of signals used in PSG method. On the other hand, the ECG and SKNA were recorded using the neuECG device. The start time of PSG and newECG recording is not exactly the same. So, the sleep scoring, SKNA and ECG signals are first synchronized. Sometimes, there are extreme artifacts in the ECG or SKNA due to motion or loose contract of ECG patch electrodes. These extreme artifacts have been discarded from the source signals, and only normal R-R intervals were considered. As some R-peaks are also mis-detected or missing as shown in FIG. 1(a), only those R-R intervals that lie within the 95% CFI of the R-R values (RR series) were considered. Further, RR and SKNA signals were segmented into a series of 30 s window called epoch.

To extract features from RR series and SKNA, the study considered a series of 5 minute length consists of 6 epochs before and 3 epochs after the current epoch as recommended in. The study extracted 14 HRV parameters and 11 SKNA parameters from each 5 minute series. The HRV parameters included:
1. MeanNN (s): The average of R-R intervals of the series measured in s.
2. SDNN (s): The standard deviation of the R-R intervals of the series (s).
3. RMSSD (s): The root mean square of the successive differences of R-R intervals in s.
4. DFA_{α1} (a.u.): The short-term detrended fluctuation scaling exponent, a nonlinear measure of the R-R intervals useful for assessing the possible nonlinear and nonstationary characteristics in the series.
5. cvNN (a.u.): The relative dispersion of R-R intervals of the series.
6. SampEn (au.): A conditional differential entropy rate that measures the complexity (or regularity in other words) of a time series. The estimation of sample entropy (SampEn) depends on the selection of m (the embedding dimension) and r (the maximum tolerance of mismatch between the corresponding elements of small vectors) for a given series length (N). It is estimated as SampEn(m, r, N)=log Pₘ−log Pₘ₊₁, where Pₘ and Pₘ₊₁, are respectively, the probability of matching vectors of length m and m+1.
7. bEn (a.u.): A proposed entropy metric called Bubble entropy, which does not require the selection of parameter r in SampEn estimation, and is less dependent on m. Unlike SampEn, it does not directly quantify conditional entropy; instead, it quantifies the entropy changes in ordering the small vectors of length m to m+1 of a series of length N. Mathematically,

$$bEn ( m , N ) = ( {H}_{swaps}^{m + 1} - {H}_{swaps}^{m} ) / log ( \frac{m + 1}{m - 1} ) ,$$

where H is the second order Rényi entropy.
8. SD1 (s): SD1 measured in (s) is the length of the semi-minor axis of the ellipse around the 95% confidence of the RR series fitted to the Poincaré plots. It measures the short-term variability of the RR series and is influenced by parasympathetic modulation.
9. SD2 (s): SD2 measured in second is the length of the semi-major axis of the ellipse around the 95% confidence of the RR series fitted to the Poincaré plots. It measures the long-term variability of the RR series and reflects sympathetic activation.
10. SD1/SD2: The ratio of SD1 to SD2 provides information about the sympatho-vagal balance of the autonomic nervous system.

The study extracted a set of four frequency domain parameters in addition to the time domain parameters. The frequency domain parameters were extracted in four frequency bands: [0.003 0.04] Hz, [0.04 0.15] Hz, and [0.15 0.4] Hz, respectively for VLF, LF, and HF.
1. VLF (%): Percentage of power in the very low frequency [0.03 0.04] Hz band.
2. LF (%): Percentage of power in the low frequency [0.04 0.15] Hz band.
3. HF (%): Percentage of power in the high frequency [0.15 0.4] Hz band.
4. LF/HF: The ratio of LF power to HF power.

The 11 features derived from the SKNA signals included:
1. aSKNA (μV): The mean of sympathetic nerve activity value per sample, measured in micro-volt (μV).
2. varSKNA (μV): The variance of SKNA (varSKNA), measured in (IN), is the signal dispersion around its mean.
3. rmsSKNA(μV): The root mean square (RMS) of the amplitude of SKNA signals, measured in μV.
4. skewSKNA: Measures skewness of the amplitude distribution of the SKNA signal.
5. kurtSKNA: Measures kurtosis i.e., the peakness of the distribution of signal's amplitude.
6. wISKNA: The waveform length (WL) of SKNA signal provides information about the shape and frequency content by measuring the cumulative length of the signal's waveform.
7. zcSKNA: The zero crossing (ZC) of SKNA counts the number of times, the SKNA crosses the zero axis, providing information about the signal's frequency.
8. sscSKNA: Counts the number of times the sign of the signal's slope (SSC) changes, providing information about the signal's rate of change.
9. wampSKNA: The Wilson amplitude (wamp) of SKNA computes the weighted sum of the absolute differences between adjacent samples that exceed a threshold value (Theta), providing information about the signal's amplitude changes over time.
10. cfSKNA: The crest factor of SKNA (cfSKNA) measures the peak-to-ratio of the SKNA signal, providing information about the signal's dynamic range.
11. dfSKNA (Hz): The dominant frequency of SKNA (dfSKNA) provides the frequency with maximum power of the SKNA signal in (Hz). The dfSKNA was estimated from the integrated SKNA signals using the periodogram method.

Feature selection involves reducing the number of variables to the input of a machine learning model to reduce computational cost and, in some cases, to improve classification performance. Statistical feature selection evaluates the relationship between each input and the target variables, and select those features which have strong relationships, which is fast and effective. However, selecting the appropriate statistical measures is sometimes challenging and critical. Instead, the study used a performance measure called Cohen's Kappa that measures the reliability of the classification model irrespective of the overall accuracy to select most important features (or input variables). The pseudocode of features selection, which the study referenced as the “Greedy Forward Approach,” is given below:
1. Divide data into 70%, 20%, and 10% for training, testing, and validation sets.
2. inputFeaturesList={All input variables}
3. selectedFeaturesList=φ
4. For each feature in the inputFeaturesList
   a. Create a classification model on the 80% training set.
   b. Evaluate Cohen's Kappa of the classification model on the test set.
   c. Add the feature for which Cohen's Kappa is maximum to the selectedFeaturesList.
   d. Exclude the selected feature from the inputFeaturesList.
5. Repeat step 4 until the inputFeaturesList is empty.

The manual sleep staging from PSG following the guidelines of AASM was considered as the gold standard in this study. The study used a support vector machine (SVM) with “rbf” kernel to classify sleep into five stages (NREM-1, NREM-2, NREM-3, REM, and WAKE), three stages (NREM, REM, and WAKE), and binary classes (SLEEP and WAKE) using all features and best relevant features. The study also evaluated the individual classification performance of HRV and SKNA parameters for binary to five stages classification problems. The best or most relevant features were selected for five stages classification problems. The data were divided into train, validation, and test sets for 70%, 10%, and 20%, respectively. The classification model was trained using K-fold (K=10) cross validation. The classification performance of the model was evaluated by Accuracy (Acc), Precision (Prec), Recall (Rec), Sensitivity (Sens), Specificity (Spec), F1 score, and Cohen's Kappa (K).

We compared both HRV and SKNA parameters to discriminate between sleep stages using paired t-test for parameters having Normal distribution or Wilcoxson Rank Sum test for those parameters which do not have Normal distribution. In addition, to observe if the addition of SKNA information with HRV parameters significantly improve the accuracy, the McNemar test was performed on the sleep stages predicted by the classification model using HRV alone and for the combination of HRV and SKNA.

The sleep staging from skin sympathetic nerve activity is possible, and the combination of SKNA with heart rate variability parameters improve sleep stages classification which is statistically significant. The SKNA itself provides sleep scoring with similar capability of HRV for binary classification problem. Therefore, this study supports that a single wearable monitor recording the ECG and SKNA signals from the standard ECG patch electrodes can be used to effectively monitor sleep patterns. These findings open the possibilities of designing wearable monitors for facilitating long-term monitoring of sleep within the home environment.

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

1. A system for identifying a plurality of sleep stages of a subject, the system comprising:
a monitor having at least one sensor configured to obtain a captured information regarding a skin nerve activity and a heart activity of the subject;
at least one processor, and
a memory coupled with the at least one processor, the memory including instructions that when executed by the at least one processor cause the at least one processor to:
determine a plurality of activity features from the captured information regarding both the skin nerve activity and the heart activity of the subject;
generate one or more signals to facilitate the determined plurality of activities being used with a machine learnable model to identify one or more sleep stages of the plurality of sleep stages; and
generate one or more signals to facilitate a communication of the identified one or more sleep stages to the subject.

2. The system of claim 1, wherein the monitor comprises an article of clothing or a clothing accessory, or adhesive patch that is wearable by the subject.

3. The system of claim 2, wherein the monitor comprises a shirt or a watch.

4. The system of claim 1, wherein the at least one sensor comprises a first sensor that obtains the captured information regarding both the skin nerve activity and the heart activity of the subject.

5. The system of claim 1, wherein the memory further includes instructions that when executed by the at least one processor cause the at least one processor to derive a first select set of activity features and a second select set of activity features from the plurality of activity features, the first select set of activity features comprising less than all of a set of the plurality of activity features related to the skin nerve activity, and the second select set of activity features comprising less than all of a set of the plurality of activity features related to the heart activity of the subject, and wherein the plurality of activity features used with a machine learnable model comprise the first and second select sets of activity features.

6. The system of claim 1, wherein the determination of the plurality of activity features from the captured information comprises:
identification of a first select set of activity features from one or more features available for identification from the captured information regarding to the skin nerve activity, the first select set of activity features being less than all activity features derivable from the captured information regarding to the skin nerve activity; and
identification of a second select set of activity features from one or more features available for identification from the captured information regarding to the heart activity, the second select set of activity features being less than all activity features derivable from the captured information regarding to the heart activity,
wherein the plurality of activity features used with a machine learnable model comprise the first and second select sets of activity features.

7. The system of claim 6, wherein the memory further includes instructions that when executed by the at least one processor cause the at least one processor to identify, based on information outputted from the machine learnable model, one or more types of activity features that are to be utilized for the first select set of activity features.

8. The system of claim 6, wherein the first select set of activity features comprise at least two of the following: aSKNA, varSKNA, rmsSKNA, skewSKNA, kurtSKNA, wISKNA, zcSKNA, sscSKNA, wampSKNA, cfSKNA, and dfSKNA.

9. The system of claim 6, wherein the memory further includes instructions that when executed by the at least one processor cause the at least one processor to identify, based on information outputted from the machine learnable model, one or more types of activity features that are to be utilized for the second select set of activity features.

10. The system of claim 6, wherein the second select set of activity features comprise at least two of the following: MeanNN, SDNN, RMSSD, DFA_{α1}, cvNN, SampEn, bEn, SD1, SD2, and SD1/SD2.

11. The system of claim 1, wherein the memory including instructions that when executed by the at least one processor cause the at least one processor to provide information for a training of the machine learnable model.

12. A method for identifying a plurality of sleep stages of a subject, the method comprising:
capturing, using at least one sensor, a captured information regarding a skin nerve activity and a heart activity of the subject;
determining a plurality of activity features from the captured information regarding both the skin nerve activity and the heart activity of the subject;
generating one or more signals to facilitate the plurality of activity features being used with a machine learnable model to identify one or more sleep stages of the plurality of sleep stages; and
generating one or more signals to facilitate a communication of the identified one or more sleep stages to the subject.

13. The method of claim 12, wherein capturing the captured information further comprises capturing the captured information using an article of clothing or a clothing accessory that is wearable by the subject.

14. The method of claim 13, wherein the article of clothing or the clothing accessory comprises a shirt or a watch.

15. The method of claim 12, wherein the at least one sensor comprises a first sensor that obtains the captured information regarding both the skin nerve activity and the heart activity of the subject.

16. The method of claim 12, wherein determining a plurality of activity features comprises:
determining a first select set of activity features from the plurality of activity features, the first select set of activity features comprising less than all of a set of the plurality of activity features related to the skin nerve activity; and
determining a second select set of activity features, the second select set of activity features comprising less than all of a set of the plurality of activity features related to the heart activity of the subject,
wherein the plurality of activity features used with a machine learnable model comprise the first and second select sets of activity features.

17. The method of claim 12, wherein determining a plurality of activity features comprises:
identifying a first select set of activity features from one or more features available for identification from the captured information regarding to the skin nerve activity, the first select set of activity features being less than all activity features derivable from the captured information regarding to the skin nerve activity; and
identifying a second select set of activity features from one or more features available for identification from the captured information regarding to the heart activity, the second select set of activity features being less than all activity features derivable from the captured information regarding to the heart activity,
wherein the plurality of activity features used with a machine learnable model comprise the first and second select sets of activity features.

18. The method of claim 17, further comprising identifying, based on information outputted from the machine learnable model, one or more types of activity features that are to be utilized for the first select set of activity features.

19. The method of claim 17, wherein the first select set of activity features comprise at least two of the following: aSKNA, varSKNA, rmsSKNA, skewSKNA, kurtSKNA, wISKNA, zcSKNA, sscSKNA, wampSKNA, cfSKNA, and dfSKNA.

20. The method of claim 17, further comprising identifying, based on information outputted from the machine learnable model, one or more types of activity features that are to be utilized for the second select set of activity features.

21. The method of claim 17, wherein the second select set of activity features comprise at least two of the following: MeanNN, SDNN, RMSSD, DFA_{α1}, cvNN, SampEn, bEn, SD1, SD2, and SD1/SD2.
